(19) United States
(12) Patent Application Publication
(10) Pub. No.: US 2024/0002893 A1
(43) Pub. Date: Jan. 4, 2024

(54) PROCESS FOR SYNTHESIZING FUNCTIONALIZED MERCAPTANS UNDER AN H2S PRESSURE

(71) Applicant: ARKEMA FRANCE, Colombes (FR)

(72) Inventors: Jean-Christophe LEC, Lacq (FR); Georges FREMY, Lacq (FR); Arnaud DESSOMMES, Lacq (FR)

(73) Assignee: ARKEMA FRANCE, Colombes (FR)

(21) Appl. No.: 18/255,803

(22) PCT Filed: Dec. 1, 2021
(86) PCT No.: PCT/FR2021/052156

§ 371 Date: Jun. 2, 2023

(30) Foreign Application Priority Data
Dec. 4, 2020 (FR) 2012665

(51) Int. Cl. C12P 13/12

(52) U.S. Cl. CPC C12P 13/12

(57) ABSTRACT

The present invention relates to a process for synthesizing a functionalized mercaptan, comprising the reaction between a compound of formula R₂—X—C*H(NR₁R₇)—(CH₂)ₙ-G (II) and H₂S in the presence of at least one enzyme chosen from sulfhydrylases; said reaction being performed in a reactor with a partial pressure of H₂S in the gas headspace of said reactor of between 0.01 and 4 bar, preferably between 0.1 and 3 bar, for example between 0.1 and 2.5 bar, and more preferentially between 0.25 and 2 bar, at the reaction temperature.

The present invention relates to a process for synthesizing functionalized mercaptans, and also to a composition making it possible in particular to implement this process.

Mercaptans are used in numerous industrial fields and many synthesis methods are known, such as the sulfhydration of alcohols, the catalytic or photochemical addition of hydrogen sulfide onto unsaturated organic compounds or the substitution, using hydrogen sulfide, of halides, epoxides or organic carbonates.

However, these processes have many drawbacks and are not always suited to the synthesis of functionalized mercaptans, that is to say mercaptans comprising at least one functional group other than the thiol group (—SH). This type of mercaptan constitutes a chemical family with a great deal of potential, especially amino acids and derivatives with a thiol function, in particular homocysteine. They may for example be useful as synthesis intermediates for the cosmetics industry. However, there is currently no effective synthesis method suited to the production of these functionalized mercaptans which is industrially viable, especially for applications falling under the field of commodity chemicals.

For instance, among the conventional chemical methods, substitution with hydrogen sulfide requires frequently high temperatures and pressures and leads to undesired by-products of olefin, ether, sulfide and/or polysulfide type. The catalytic or photochemical addition of hydrogen sulfide onto unsaturated compounds is generally performed under slightly milder conditions but likewise leads to many by-products formed by isomerization of the starting material, by non-regioselective addition or by double addition leading to the production of sulfides and/or polysulfides.

These conventional synthesis methods therefore require operating conditions which are too harsh for compounds such as functionalized mercaptans and result in the coproduction of a significant amount of sulfides and/or polysulfides which are difficult to upgrade.

It is a known alternative to the chemical routes to synthesize functionalized mercaptans via the biological route. For example, cysteine is currently produced biologically by a fermentation route (Maier T., 2003. Nature Biotechnology, 21: 422-427). These biological routes are gentler and better suited to multifunctional molecules. However, these biological routes often have low yields and/or are not readily transposable to or viable on an industrial scale. Furthermore, here again, the production of the mercaptan of interest is accompanied by the corresponding sulfides and/or polysulfides such as disulfides (see for example international application WO 2012/053777).

There is therefore a need for an improved process for synthesizing functionalized mercaptans, in particular by the biological route.

In particular, there is a need for a process for synthesizing functionalized mercaptans which makes it possible to obtain a satisfactory yield, or even a yield of at least 20%, preferably of at least 60%, more preferably of at least 80%, more preferentially of at least 90%.

There is also a need for a process for synthesizing functionalized mercaptans which is viable on an industrial scale, with gentle operating conditions.

One object of the present invention is to provide an improved process for synthesizing a functionalized mercaptan, in particular having an improved yield, or even a yield of at least 20%, preferably of at least 60%, more preferably of at least 80%, more preferentially of at least 90%.

Another object of the present invention is to provide an industrial process with operating conditions which are gentle and adapted to the synthesis of a multifunctional mercaptan.

Another object of the present invention is to provide a process which avoids the use of hydrosulfide salt and/or of sulfide salt as reagent, and thus is more environmentally friendly. The present invention fully or partly fulfils the objects above.

According to the present invention, the functionalized mercaptans of formula (I) as defined below, in particular L-homocysteine, are advantageously synthesized by reaction between compounds of formula (II) and H₂S, in the presence of a sulfhydrylase enzyme, under a particular range of partial pressure of H₂S in the reactor where said reaction takes place. In particular, said partial pressure of H₂S is between 0.01 and 4 bar, for example between 0.01 and 3 bar, preferably between 0.1 and 3 bar, for example between 0.1 and 2.5 bar, and more preferentially between 0.25 and 2 bar.

The present inventors have thus discovered that the conversion of the compounds of formula (II) into functionalized mercaptans of formula (I) is strongly dependent on the partial pressure of H₂S in the reactor. Surprisingly, the present inventors have discovered that in a specific range of partial pressure of H₂S in the reactor, a conversion and/or a yield of at least 20%, preferably of at least 60%, more preferably of at least 80%, more preferentially of at least 90%, is obtained. For example, the conversion and/or the yield is (are) between 80% and 100%, or even between 90% and 100%. In particular, the conversion and/or the yield is (are) 100%. Indeed, contrary to what was expected, increasing the partial pressure of H₂S in the reactor beyond a certain limit does not make it possible to increase the conversion and/or the yield of the reaction but limits or even inhibits the latter. It could be expected that the more the partial pressure of H₂S increases in the reactor, the more the amount of H₂S increases in the reaction medium (in particular in dissolved form in a liquid reaction medium), thus promoting the reaction. However, too high a partial pressure of H₂S is in reality detrimental to the reaction.

Furthermore, the specific range of partial pressure of H₂S in the reactor according to the invention allows rapid reaction kinetics. For example, 100% yield may be achieved in one hour. The reaction time may thus be between 0.15 h and 10 h, for example between 0.25 h and 4 h, preferably between 0.5 h and 1 h. it is also observed that the process according to the invention makes it possible to obtain better yields than a process that uses hydrosulfide salts and/or sulfide salts as reagents. The use of hydrogen sulfide thus makes it possible to limit, or even to simplify the steps of purifying and managing the effluents which are necessary when such salts are used. The process according to the invention is therefore more environmentally friendly.

The present invention thus relates to a process for synthesizing at least one functionalized mercaptan of the following general formula (I):

R₂—X—C*H(NR₁R₇)—(CH₂)ₙ—SH (I)

in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from —C(═O)—, —CH₂— or —CN;
R₂ is:
(i) either absent when X represents —CN,
(ii) or a hydrogen atom,
(iii) or —OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
(iv) or —NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;

n is equal to 1 or 2; and * represents an asymmetric carbon;

said process comprising the stages of:
a) provision of at least one compound of the following general formula (II):

R₂—X—C*H(NR₁R₇)—(CH₂)ₙ-G (II)

in which *, R₁, R₂, R₇, X and n are as defined for formula (I) and

G represents either (i) R₆—C(O)—O—, or (ii) (R₇O)(R₈O)—P(O)—O—, or (iii) R₉O—SO₂—O—; with R₆ being a hydrogen atom or a linear, branched or cyclic, saturated or unsaturated hydrocarbon chain of 1 to 20 carbon atoms which may comprise one or more aromatic groups and may be substituted by one or more groups chosen from —OR₁₀, (═O), —C(O)OR₁₁, —NR₁₂R₁₃; R₁₀, R₁₁, R₁₂ and R₁₃ being independently chosen from:

H or a linear, branched or cyclic, saturated or unsaturated hydrocarbon chain of 1 to 20 carbon atoms;

R₇ and R₈, which are identical or different, being a proton, an alkali metal, an alkaline earth metal or an ammonium;

R₉ being chosen from a proton, an alkali metal, an alkaline earth metal or an ammonium;
b) provision of H₂S;
c) reaction between said at least one compound of formula (II) and H₂S in the presence of at least one enzyme chosen from sulfhydrylases, preferably a sulfhydrylase associated with said compound of formula (II);

said reaction being performed in a reactor with a partial pressure of H₂S in the gas headspace of said reactor of between 0.01 and 4 bar, for example between 0.01 and 3 bar, preferably between 0.1 and 3 bar, for example between 0.1 and 2.5 bar, and more preferentially between 0.25 and 2 bar, at the reaction temperature;
d) obtaining of at least one functionalized mercaptan of formula (I);
e) optional separation of said at least one functionalized mercaptan of formula (I) which is obtained in stage d); and
f) optional additional functionalization and/or optional deprotection of the functionalized mercaptan of formula (I) which is obtained in stage d) or e); and wherein stages a) and b) are optionally performed simultaneously.

The expression “between X and X” includes the limits mentioned unless indicated otherwise. An unsaturated hydrocarbon chain is understood to be a hydrocarbon chain comprising at least one double or triple bond between two carbon atoms.

A heteroatom is understood in particular to be an atom chosen from O, N, S, P and halogens. An inert gas is understood in particular to be any gas having little or no reactivity in the context of the process according to the invention. Mention may be made, by way of example, of dinitrogen, argon or methane, preferably dinitrogen.

A reaction medium (or mixture) is understood in particular to be a medium comprising at least one compound of formula (II), H₂S, and said at least one sulfhydrylase.

Said reaction medium may thus comprise:
at least one compound of formula (II) as defined below,
H₂S,
at least one sulfhydrylase as defined below,
optionally its cofactor as defined below,
optionally a base as defined below, and
optionally a solvent, preferably water.

Preferably, the reaction medium is liquid, for example in the form of an aqueous solution, in particular under the temperature and pressure conditions of stage c).

The H₂S is in gaseous form, in particular under the temperature and pressure conditions of stage c). In particular, it is understood that a portion of the H₂S is dissolved in the reaction medium in order for the reaction of stage c) to take place whilst the other portion is in gaseous form in the gas headspace of the reactor, at said partial pressure.

The “gas headspace” is understood to mean the space in the reactor located above the reaction medium, preferably above the liquid reaction medium. More particularly, the “gas headspace” is understood to mean the space located between the surface of the liquid reaction medium and the top of the reactor (upper portion of the reactor comprising the gas phase when the lower portion of the reactor comprises a liquid phase). The gas headspace comprises in particular a gas phase comprising H₂S at said partial pressure.

The reaction medium and the H₂S are in particular introduced into the reactor in amounts such that a gas headspace is located above the reaction medium contained in the reactor.

Alternatively, stage c) may be described as follows:
c) reaction between said at least one compound of formula (II) and H₂S in the presence of at least one enzyme chosen from sulfhydrylases, preferably a sulfhydrylase associated with said compound of formula (II);

said reaction being performed in a reactor with a partial pressure of H₂S above the reaction medium of between 0.01 and 4 bar, for example between 0.01 and 3 bar, preferably between 0.1 and 3 bar, for example between 0.1 and 2.5 bar, and more preferentially between 0.25 and 2 bar, at the reaction temperature.

According to one embodiment, said partial pressure of H₂S corresponds to the total pressure of the gas phase present in the gas headspace (i.e. only H₂S is present in the gas headspace of the reactor).

According to one embodiment, said partial pressure of H₂S may be kept constant throughout the entire duration of stage c). This may be obtained by continuous introduction of H₂S into the reactor or by regular or irregular isolated additions of H₂S into the reactor, during stage c). Indeed, since the H₂S is consumed during the reaction, it is thus possible to compensate for the reduction in the partial pressure of H₂S.

According to another embodiment, said partial pressure of H₂S can be achieved before or during stage c), then the introduction of H₂S into the reactor is stopped. The partial pressure of H₂S therefore decreases during stage c), preferably until the shutdown of the reaction.

The partial pressure of H₂S can be controlled throughout stage c), in particular by any known technique, for example using a manometer. H₂S can be added so that a state of equilibrium between the liquid phase (reaction medium) and the gas phase (comprising H₂S at said partial pressure) is reached in the reactor.

Preferably, the total pressure of the gas phase in the gas headspace (for example the pressure of H₂S when the latter is the only gas or the total pressure of the mixture of H₂S and of an inert gas) corresponds approximately to atmospheric pressure (approximately 1.01325 bar). It is also possible to choose to work at underpressure or overpressure relative to atmospheric pressure depending on the desired operating conditions.

Mention may for example be made of the following methods.
According to one embodiment, a vacuum is created in the reactor then H₂S at the partial pressure according to the invention is introduced. For example, a vacuum is drawn down to −1 bar then an H₂S pressure of 0.25 bar is applied.
According to another embodiment, the following stages are performed:
   purging the headspace of the reactor with an inert gas, such as N₂; then
   creating a partial vacuum; then
   introducing the H₂S at the partial pressure according to the invention.
   For example, after purging the headspace of the reactor with N₂, drawing a vacuum to −0.25 bar then adding 0.25 bar of H₂S.
According to another embodiment, it is possible to introduce into the reactor a mixture of inert gas, such as N₂, and of H₂S at the partial pressure according to the invention. For example, it is possible to introduce a mixture of 0.25 bar of H₂S and 0.75 bar of N₂.
According to another embodiment, it is possible to introduce into the reactor an inert gas (purging of the gas headspace), such as N₂, then to add H₂S at the partial pressure according to the invention. For example, it is possible to apply pressure of 1 bar of N₂ then to add 0.25 bar of H₂S.

The temperature during stage c) may be between 10° C. and 60° C., preferably between 20° C. and 40° C., and more particularly between 25° C. and 40° C.

The reactor used for stage c) may be of any type. It is preferably chosen from plug-flow reactors or continuous reactors, preferably that are stirred and/or have recirculation of the gas phase and/or recirculation of the liquid phase. Preferably, said reactor enables a recirculation (or recycling) of the gas phase present in the gas headspace.

Stages a) and b) may be simultaneous or carried out in any order.

Said reaction medium can be prepared by adding said compound of formula (II), said sulfhydrylase and optionally its cofactor in any order. It is preferable to introduce the H₂S next. This makes it possible in particular to more easily manage the H₂S pressure introduced into the reactor.

Preferably, the compound of formula (II) and/or the sulfhydrylase is(are) in the form of a solution, more preferentially in the form of an aqueous solution.

The H₂S may be introduced into the reactor by any known method and in particular by bubbling into the reaction medium, preferably by bubbling into the reaction medium from the bottom of the reactor. The bubbling can be effected by mixing H₂S with an inert gas, for example dinitrogen, argon or methane, preferably dinitrogen. Preferentially, the H₂S is introduced pure (without being mixed with another gas). The H₂S may also be introduced via the headspace of the reactor and for example may then equilibrate with the reaction medium, the reaction medium preferably being stirred.

Preferably, the H₂S is in excess, preferably in molar excess, relative to the compound of formula (II), preferably during stage c) and more preferentially during the entire duration of stage c). The H₂S can therefore be in a superstoichiometric amount relative to the amount of the compound of formula (II), preferably during stage c) and more preferentially during the entire duration of stage c).

In particular, the molar ratio H₂S/compound of formula (II) is between 1.1 and 20, preferably between 1.1 and 10, preferentially between 2 and 8, for example between 3.5 and 8, and even more preferentially between 3.5 and 5, preferably during stage c) and more preferentially during the entire duration of stage c). Said ratio may be kept constant during the entire duration of stage c).

Stage c) can be carried out in solution, in particular in aqueous solution. For example, the solution comprises between 50% and 99% by weight of water, preferably between 75% and 97% by weight of water, relative to the total weight of the solution.

The pH of the reaction medium in stage c) can be between 4 and 9, for example between 5 and 8, preferably between 6 and 7.5, and more particularly between 6.2 and 7.2, in particular when the reaction medium is an aqueous solution.

The pH can in particular be adjusted within the abovementioned ranges according to the operating optimum of the chosen sulfhydrylase. The pH can be determined by conventionally known methods, for example with a pH probe. The pH can in particular be adjusted by adding a base, preferably throughout the reaction of stage c). Any type of base may be used, preferably a base comprising a sulfur atom. A base is understood in particular to be a compound or a mixture of compounds having a pH of greater than 7, preferably between 8 and 14.

The base can be chosen from hydrosulfide salts and/or sulfide salts, sodium hydroxide, potassium hydroxide or ammonia. The preferred base is ammonium hydrosulfide (NH₄SH). The hydrosulfide and/or sulfide salt can be chosen from the group consisting of: ammonium hydrosulfide, alkali metal hydrosulfides, alkaline earth metal hydrosulfides, alkali metal sulfides and alkaline earth metal sulfides.

Alkali metals are understood to be lithium, sodium, potassium, rubidium and caesium, preferably sodium and potassium.

Alkaline earth metals are understood to be beryllium, magnesium, calcium, strontium and barium, preferably calcium.

In particular, the hydrosulfide salt and/or sulfide salt can be chosen from the group consisting of:
ammonium hydrosulfide NH₄SH, sodium hydrosulfide NaSH, potassium hydrosulfide KSH, calcium hydrosulfide Ca(SH)₂, sodium sulfide Na₂S, ammonium sulfide (NH₄)₂S, potassium sulfide K2S and calcium sulfide CaS. The preferred hydrosulfide is ammonium hydrosulfide NH₄SH.

The base can be added at a concentration of between 0.1 and 10 M, preferably between 0.5 and 10 M, more preferably between 0.5 and 5 M. Use will in particular be made of concentrated bases so as to limit the dilution of the reaction medium when adding the base.

Stage c) may be performed batchwise, semi-continuously or continuously.

Stage c) Carried Out Essentially in the Absence of Oxygen:

Oxygen is understood in particular to mean dioxygen O₂.

Preferably, stage c) is carried out essentially in the absence of oxygen, or even in the absence of oxygen. When stage c) is carried out essentially in the absence of oxygen (or even in the absence of oxygen O₂), this makes it possible if need be to limit (or even to prevent) the coproduction of sulfides and/or polysulfides, in particular of disulfides, which are unwanted by-products (cf. application FR2007577).

More particularly, the expression “essentially in the absence of oxygen” is understood to mean that an amount of oxygen may remain in the reaction medium and/or in the gas phase (contained in the gas headspace of the reactor), such that the amount of sulfides and/or polysulfides produced is less than or equal to 5% by weight relative to the total weight of the compound of formula (I) produced.

For example, the expression “essentially in the absence of oxygen” is understood to mean that the reaction medium contains less than 0.0015% oxygen (preferably strictly less than 0.0015%) by weight relative to the total weight of the reaction medium and/or that the gas phase (contained in the gas headspace) contains less than 21% oxygen (preferably strictly less than 21%) by volume relative to the total volume of said gas phase.

Thus, the reaction medium may contain between 0 and 0.0015% oxygen (preferably strictly less than 0.0015%) by weight relative to the total weight of the reaction medium and/or the gas phase (contained in the gas headspace) may contain between 0 and 21% oxygen (preferably strictly less than 21%) by volume relative to the total volume of the gas phase. In particular, the amount of oxygen in the reaction medium and/or in the gas phase (contained in the gas headspace) is such that the amount of sulfides and/or polysulfides produced is less than or equal to 5% by weight relative to the total weight of the compound of formula (I) produced. For example, stage c) may be carried out in a closed reactor (i.e. without a supply of oxygen from the air).

Quite preferably, the gas phase (contained in the gas headspace) does not comprise oxygen. Preferably, the gas phase (contained in the gas headspace) does not comprise oxygen and the reaction mixture comprises between 0 and 0.0015% oxygen (preferably strictly less than 0.0015%) by weight relative to the total weight of the reaction mixture. This is because the O₂/H₂S mixture may present an explosive risk which, obviously implies a risk for the safety of the operators.

More particularly, when stage c) is also carried out essentially in the absence of oxygen (or even in the absence of oxygen) this makes it possible if need be to produce L-homocysteine while limiting (or even preventing) the coproduction of L-homocystine and/or of L-homocysteine sulfide (also referred to as 4,4′-sulfanediylbis(2-aminobutanoic acid)/L-homolanthionine), unwanted by-products.

L-homocysteine sulfide has the following formula:

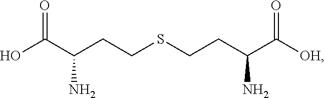 [SMILES: N[C@@H](CCSCC[C@H](N)C(=O)O)C(=O)O]

L-homocystine has the following formula:

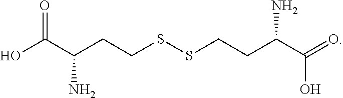 [SMILES: N[C@@H](CCSSCC[C@H](N)C(=O)O)C(=O)O]

Conventional methods can be used for performing stage c), essentially in the absence of oxygen or even in the absence of oxygen.

According to one embodiment, prior to stage c) the oxygen is removed from the reaction medium, for example by degassing.

According to another embodiment, prior to stage c), the oxygen is removed separately from each of the components or from the mixture of at least two thereof that are going to form the reaction medium. For example, each of the solutions comprising the compound of formula (II), the sulfhydrylase and optionally the solvent, are degassed.

It is also possible to remove the oxygen from the gas phase of the headspace of the reactor, preferably by degassing.

The reactor can also be inertized with an inert gas such as dinitrogen, argon or methane, preferably dinitrogen.

Various techniques may also be combined with each other.

Preferably, the substantial or even total absence of oxygen is achieved in the following way:
the reactor is inertized with an inert gas such as dinitrogen, argon or methane, preferably dinitrogen; and
each of the solutions comprising the compound of formula (II), the sulfhydrylase and optionally the solvent, are degassed.

Industrial degassing methods are well known and mention may for example be made of the following:
pressure reduction (vacuum degassing),
thermal regulation (increasing the temperature for an aqueous solvent and lowering the temperature for an organic solvent),
membrane degassing,
degassing by alternating freeze-pump-thaw cycles,
degassing by sparging with an inert gas (for example argon, dinitrogen or methane).

According to one embodiment, in stage c) the oxygen is neither present in a form dissolved in a liquid (in particular in the reaction medium) nor in gaseous form (in particular in said gaseous phase).

The separation stage e) can be performed according to any technique known to a person skilled in the art. In particular, when the final product is a solid:
by extraction and/or decantation with a solvent which is immiscible in the reaction medium, followed by an evaporation of said solvent;
by precipitation (by partial evaporation of the solvents or by addition of a solvent in which the compound of interest is less soluble). This precipitation is generally followed by a stage of filtration according to any method known to a person skilled in the art. The final product can then be dried; or
by selective precipitation via adjustment of the pH as a function of the respective solubilities of the different compounds.

Homocysteine may in particular be recovered in solid form.

When the final product is in liquid form, the separation can be performed by distillation or by distillation or evaporation preceded by a liquid/liquid extraction.

Stage f) of additional functionalization and/or optional deprotection can make it possible to obtain additional chemical functions and/or to deprotect certain chemical functions by conventional methods. For example, if X—R₂ represents a carboxyl functional group, the latter can be esterified, reduced to an aldehyde, reduced to an alcohol and then esterified, amidated, nitrilated or others. All the functional groups can be obtained and/or deprotected by a person skilled in the art depending on the final use which is intended for said functionalized mercaptan of formula (I).

Thus, the functionalized mercaptan of formula (I) obtained on conclusion of stage d) or e) may be subjected to one or more additional chemical reactions in order to obtain one or more mercaptan derivatives with different functionalities, said chemical reactions being reactions that are well known.

Functionalized mercaptans of general formula (I):

The process according to the invention is targeted at obtaining functionalized mercaptans of the following general formula (I):

R₂—X—C*H(NR₁R₇)—(CH₂)ₙ—SH (I)

in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from —C(═O)—, —CH₂— or —CN;
R₂ is:
(i) either absent when X represents —CN,
(ii) or a hydrogen atom,
(iii) or —OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
(iv) or —NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon.

These mercaptans are referred to as functionalized because, in addition to the chemical function —SH, they also comprise at least one amine-type function —NR₁R₇.

Preferably, n is equal to 2.

Preferably, X is —C(═O)—.

Preferably, R₂ is —OR₃ with R₃ as defined above. R₃ may in particular be a hydrogen atom or a linear or branched, saturated hydrocarbon chain of 1 to 10 carbon atoms, preferably of 1 to 5 carbon atoms. In particular, R₃ is H.

R₁ and R₇, which are identical or different, are preferably a hydrogen atom or a linear or branched, saturated hydrocarbon chain of 1 to 10 carbon atoms, preferably of 1 to 5 carbon atoms. Preferably, R₁ and R₇ are H.

In particular, X is —C(═O)— and R₂ is —OR₃ with R₃ as defined above.

The functionalized mercaptans of formula (I) may be chosen from the group consisting of homocysteine, cysteine, and derivatives of these.

In particular, the functionalized mercaptans of formula (I) are L-homocysteine and L-cysteine.

A preferred functionalized mercaptan of formula (I) is homocysteine, and very particularly L-homocysteine of the following formula:

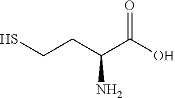

For L-homocysteine, n is equal to 2, X is —C(═O)—, R₂ is —OR₃ with R₃ being H and R₁ and R₇ are H.

It has been observed that the configuration of the asymmetric carbon atoms is retained throughout the reaction of stage c). Therefore, the functionalized mercaptan of formula (I) obtained according to the process of the invention may be enantiomerically pure.

The functionalized mercaptans of formula (I) are chiral compounds. In the present description, when the enantiomeric form is not specified, the compound is included whatever its enantiomeric form.

According to one embodiment, the reaction medium at the end of stage c) does not comprise sulfide or polysulfide and in particular does not comprise sulfide or polysulfide corresponding to the functionalized mercaptan of formula (I) obtained. For example, the reaction medium at the end of stage c) comprises less than 10 mol %, preferably less than 5 mol %, of sulfides and polysulfides relative to the total number of moles of compound of formula (II) converted into compound of formula (I).

Sulfide is understood in particular to be the sulfide corresponding to the compound of formula (I) which is that of the following formula (III):

R₂—X—C*H(NR₁R₇)—(CH₂)ₙ—S—(CH₂)ₙ—(NR₁R₇)C*H—X—R₂ (III)

with *, R₁, R₂, R₇, X and n as defined above.

Polysulfide is understood in particular to be the polysulfide corresponding to the compound of formula (I) which is that of the following formula (IV):

R₂—X—C*H(NR₁R₇)—(CH₂)ₙ—(S)ₘ—(CH₂)ₙ—(NR₁R₇)C*H—X—R₂ (IV)

with *, R₁, R₂, R₇, X and n as defined above and m being an integer between 2 and 6, limits included, for example m is equal to 2 or 3.

Preferably, m is equal to 2 (which corresponds to a disulfide).

In particular, the reaction medium at the end of stage c) does not comprise L-homocysteine sulfide or L-homocystine when the compound of formula (I) is L-homocysteine.

Preferably, following the reaction of the compound of formula (II) with H₂S during stage c) the following are obtained: a functionalized mercaptan of formula (I) as defined above and a compound of formula (V) GH with G as defined below, namely a compound of the type: (i′) R₆—C(O)—OH, (ii′) (R₇O)(R₈O)—P(O)—OH, or (iii′) R₉O—SO₂—OH; with R₆, R₇, R₈ and R₉ as defined below. In particular, when the compound (II) is O-acetyl-L-homoserine, L-homocysteine and acetic acid are obtained. The compounds of formula (V) may be responsible for the acidification of the reaction medium during stage c). Therefore, it is possible to maintain the pH of the reaction medium between 4 and 9, for example between 5 and 8, preferably between 6 and 7.5, and more particularly between 6.2 and 7.2, in particular during stage c) as mentioned above and in particular by the addition of a base as defined above.

Compounds of General Formula (II):

For the compounds of the following general formula (II):

R₂—X—C*H(NR₁R₇)—(CH₂)ₙ-G (II)

*, R₁, R₂, R₇, X and n are as defined above for the compounds of formula (I), and G represents either (i) R₆—C(O)—O—, or (ii) (R₇O)(R₈O)—P(O)—O—, or (iii) R₉O—SO₂—O—; with R₆ being a hydrogen atom or a linear, branched or cyclic, saturated or unsaturated hydrocarbon chain of 1 to 20, preferably 1 to 10, carbon atoms which may comprise one or more aromatic groups and may be substituted by one or more groups chosen from —OR₁₀, (═O), —C(O)OR₁₁, and —NR₁₂R₁₃;

R₁₀, R₁₁, R₁₂ and R₁₃ being independently chosen from:

H or a linear, branched or cyclic, saturated or unsaturated hydrocarbon chain of 1 to 20, preferably 1 to 10, carbon atoms;

R₇ and R₈, which are identical or different, being a proton, an alkali metal, an alkaline earth metal or an ammonium, preferably a proton or an alkali metal and more particularly H⁺ or Na⁺;

R₉ is chosen from a proton, an alkali metal, an alkaline earth metal or an ammonium, preferably a proton or an alkali metal and more particularly a proton H⁺ or Na⁺;

In particular, G represents either R₆—C(O)—O— or R₉O—SO₂—O—; preferably G is R₆—C(O)—O—.

In particular, R₆ is a hydrogen atom or a linear or branched, saturated or unsaturated hydrocarbon chain of 1 to 10, preferably 1 to 5, carbon atoms which may be substituted by one or more groups chosen from —OR₁₀, (═O) and —C(O)OR₁₁; R₁₀ and R₁₁ being independently chosen from:

H or a linear or branched, saturated or unsaturated hydrocarbon chain of 1 to 10, preferably 1 to 5, carbon atoms.

More particularly, R₁₀ and R₁₁ are H. In particular, R₁₂ and R₁₃ are H.

Aromatic group is understood preferentially to be the phenyl group.

The compound of general formula (II) is in particular a derivative of serine (when n is equal to 1) or homoserine (when n is equal to 2), in particular of L-serine or of L-homoserine. It may for example be chosen from the group consisting of:

O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine, O-sulfato-L-homoserine, O-phospho-L serine, O-succinyl-L-serine, O-acetyl-L-serine, O-acetoacetyl-L-serine, O-propio-L-serine, O-coumaroyl-L-serine, O-malonyl-L-serine, O-hydroxymethylglutaryl-L-serine, O-pimelyl-L-serine and O-sulfato-L-serine.

More particularly, it may be chosen from the group consisting of:

O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine and O-sulfato-L-homoserine.

The compound of general formula (II) may be chosen from the group consisting of:

O-phospho-L-homoserine, O-succinyl-L-homoserine, O-acetyl-L-homoserine, O-sulfato-L-homoserine and O-propio-L-homoserine.

The compound of general formula (II) may be chosen from the group consisting of:

O-phospho-L-homoserine, O-succinyl-L-homoserine, O-acetyl-L-homoserine.

The compound of formula (II) which is very particularly preferred is O-acetyl-L-homoserine (OAHS), a compound for which n is equal to 2, X is —C(═O)—, R₂ is —OR₃ with R₃ being H, R₁ and R₇ are H and G is —O—C(O)—R₆ with R₆ being a methyl.

The compounds of formula (II) are either commercially available or obtained via any technique known to a person skilled in the art.

They may be obtained by a fermentation process from a source of hydrocarbon and nitrogen, for example as described in the application WO 2008/013432.

They may be obtained, for example, by fermentation of a renewable starting material. The renewable starting material may be chosen from glucose, sucrose, starch, molasses, glycerol and bioethanol, preferably glucose.

The L-serine derivatives may also be produced from the acetylation of L-serine, the L-serine itself possibly being obtained by fermentation of a renewable starting material. The renewable starting material may be chosen from glucose, sucrose, starch, molasses, glycerol and bioethanol, preferably glucose.

The L-homoserine derivatives may also be produced from the acetylation of L-homoserine, the L-homoserine itself possibly being obtained by fermentation of a renewable starting material.

The renewable starting material may be chosen from glucose, sucrose, starch, molasses, glycerol and bioethanol, preferably glucose.

Sulfhydrylases:

The reaction between said at least one compound of formula (II) and H₂S is performed in the presence of at least one enzyme chosen from sulfhydrylases, preferably a sulfhydrylase associated with said compound of formula (II). The sulfhydrylase associated with a compound of formula (II) is easily identifiable since it shares the same name, for example O-acetyl-L-homoserine sulfhydrylase (OAHS Sulfhydrylase) is associated with O-acetyl-L-homoserine. The sulfhydrylase in particular enables catalysis of the reaction between said compound of formula (II) and H₂S (enzymatic reaction). “Catalyst” is understood generally to be a substance which accelerates a reaction and which is unchanged at the end of this reaction. The sulfhydrylase, and optionally its cofactor, can be used in a catalytic amount. “Catalytic amount” is understood in particular to be an amount sufficient to catalyse a reaction. More particularly, a reagent used in a catalytic amount is used in a smaller amount, for example between around 0.01% and 20% by weight, relative to the amount by weight of a reagent used in stoichiometric proportion.

Said sulfhydrylase enzyme preferably belongs to the transferases class, notably designated by the EC 2.X.X.XX (or noted EC 2) classification. The EC classification for «Enzyme Commission numbers» is widely used and can be found on the website https://enzyme.expasy.org/. In particular, said enzyme is chosen among sulfhydrylases of the EC 2.5.X.XX class (or noted EC 2.5), meaning transferases transferring alkyl or aryl group, other than methyl group.

The sulfhydrylases are in particular of the class EC 2.5.1.XX (with XX varying depending on the substrate of the enzyme).

For example:
O-acetylhomoserine sulfhydrylase is of type EC 2.5.1.49.
O-phosphoserine sulfhydrylase is of type EC 2.5.1.65.
O-succinylhomoserine sulfhydrylase is of type EC 2.5.1.49.

For example:
O-acetyl-L-homoserine sulfhydrylase is of type EC 2.5.1.49.
O-phospho-L-serine sulfhydrylase is of type EC 2.5.1.65.
O-succinyl-L-homoserine sulfhydrylase is of type EC 2.5.1.49.

Thus, in particular when the compound of formula (II) is a derivative of L-homoserine or of L-serine, the sulfhydrylase used can be chosen from O-phospho-L-homoserine sulfhydrylase, O-succinyl-L-homoserine sulfhydrylase, O-acetyl-L-homoserine sulfhydrylase, O-acetoacetyl-L-homoserine sulfhydrylase, O-propio-L-homoserine sulfhydrylase, O-coumaroyl-L-homoserine sulfhydrylase, O-malonyl-L-homoserine sulfhydrylase, O-hydroxymethylglutaryl-L-homoserine sulfhydrylase, O-pimelyl-L-homoserine sulfhydrylase, O-sulfato-L-homoserine sulfhydrylase, O-phospho-L-serine sulfhydrylase, O-succinyl-L-serine sulfhydrylase, O-acetyl-L-serine sulfhydrylase, O-acetoacetyl-L-serine sulfhydrylase, O-propio-L-serine sulfhydrylase, O-coumaroyl-L-serine sulfhydrylase, O-malonyl-L-serine sulfhydrylase, O-hydroxymethylglutaryl-L-serine sulfhydrylase, O-pimelyl-L-serine sulfhydrylase and O-sulfato-serine sulfhydrylase.

More particularly, the sulfhydrylase used can be chosen from O-phospho-L-homoserine sulfhydrylase, O-succinyl-L-homoserine sulfhydrylase, O-acetyl-L-homoserine sulfhydrylase, O-acetoacetyl-L-homoserine sulfhydrylase, O-propio-L-homoserine sulfhydrylase, O-coumaroyl-L-homoserine sulfhydrylase, O-malonyl-L-homoserine sulfhydrylase, O-hydroxymethylglutaryl-L-homoserine sulfhydrylase, O-pimelyl-L-homoserine sulfhydrylase, O-sulfato-L-homoserine sulfhydrylase.

In particular, the sulfhydrylase can be chosen from O-phospho-L-homoserine sulfhydrylase, O-succinyl-L-homoserine sulfhydrylase, O-acetyl-L-homoserine sulfhydrylase, O-sulfato-L-homoserine sulfhydrylase and O-propio-L-homoserine sulfhydrylase.

The sulfhydrylase can be chosen from O-phospho-L-homoserine sulfhydrylase, O-succinyl-L-homoserine sulfhydrylase and O-acetyl-L-homoserine sulfhydrylase.

Very particularly preferably, the enzyme is O-acetyl-L-homoserine sulfhydrylase (OAHS Sulfhydrylase).

Said sulfhydrylase, and in particular the O-acetyl-L-homoserine sulfhydrylase, may originate from or be derived from the following bacterial strains: Pseudomonas sp., Chromobacterium sp., Leptospira sp. ou Hyphomonas sp.

The sulfhydrylases can function, as is perfectly known to a person skilled in the art, in the presence of a cofactor such as pyridoxal 5′-phosphate (also known as PLP) or one of its analogues, preferably pyridoxal 5′-phosphate.

Among the analogues of the cofactor pyridoxal phosphate, mention may be made of α⁵-pyridoxalmethylphosphate, 5′-methylpyridoxal-P, pyridoxal 5′-sulfate, α⁵-pyridoxalacetic acid or any other known derivative (Groman et al., Proc. Nat. Acad. Sci. USA Vol. 69, No. 11, pp. 3297−3300, November 1972).

According to one embodiment, a cofactor of the sulfhydrylase can be added to the reaction medium. Thus, a cofactor of the sulfhydrylase, for example pyridoxal 5′-phosphate, may be provided prior to stage c), or may be added during stage c). When stage c) is performed in aqueous solution, the enzyme and optionally its cofactor can be dissolved beforehand in water before being added to said solution.

According to another embodiment, cells, for example bacterial cells or other cells, may produce or even overproduce said cofactor while simultaneously expressing or overexpressing the sulfhydrylase enzyme, so as to avoid a step of supplementing said cofactor.

According to one embodiment, the sulfhydrylase, and optionally its cofactor, are:
either in isolated and/or purified form, for example in aqueous solution;

The isolation and/or the purification of said produced enzyme can be carried out by any means known to a person skilled in the art. It may for example involve a technique chosen from electrophoresis, molecular sieving, ultracentrifugation, differential precipitation, for example with ammonium sulfate, ultrafiltration, membrane or gel filtration, ion exchange, separation via hydrophobic interactions, or affinity chromatography, for example of IMAC type.
or present in a crude extract, that is to say in an extract of milled cells (lysate); the enzyme of interest may or may not be overexpressed in said cells, hereinafter denoted host cells. The host cell may be any host cell appropriate for the production of the enzyme of interest from the expression of the corresponding coding gene. This gene will then be either located in the genome of the host or carried by an expression vector.

For the purposes of the present invention, “host cell” is in particular understood to be a prokaryotic or eukaryotic cell. Host cells commonly used for the expression of recombinant or non-recombinant proteins include in particular cells of bacteria such as Escherichia coli or Bacillus sp., or Pseudomonas, cells of yeast such as Saccharomyces cerevisiae or Pichia pastoris, cells of fungi such as Aspergillus niger, Penicillium funiculosum or Trichoderma reesei, insect cells such as Sf9 cells, or else mammal (in particular human) cells such as the HEK 293, PER-C6 or CHO cell lines.

Preferably, the enzyme of interest and optionally the cofactor are expressed in the bacterium Escherichia coli. Preferentially, the enzyme of interest is expressed within a strain of Escherichia coli such as for example Escherichia coli BL21(DE3).

The cell lysate can be obtained according to various known techniques such as sonication, pressure (French press), via the use of chemical agents (e.g. xylene, triton), etc. The lysate obtained corresponds to a crude extract of milled cells.
or present in whole cells. For this, the same techniques as above can be used, without performing the cell Iysis step.

According to one embodiment, the amount of biomass expressing the sulfhydrylase enzyme, relative to the mass of the compound of formula (II), is between 0.1% and 10% by weight, preferably between 1% and 5% by weight, and/or the amount of cofactor relative to the compound of formula (II) is between 0.1% and 10% by weight, preferably between 0.5% and 5% by weight.

The reaction medium may also comprise:
optionally one or more solvents chosen from water, buffers such as phosphate buffers, Tris-HCl, Tris base, ammonium bicarbonate, ammonium acetate, HEPES (4-(2-hydroxyethyl)−1-piperazineethanesulfonic acid), CHES (N-cyclohexyl-2-aminoethanesulfonic acid), or salts such as sodium chloride, potassium chloride, or media thereof;
optionally additives such as surfactants, in order in particular to promote the solubility of one or more reagents or substrates.

The various components which can be used for the reaction of stage c) above are readily commercially obtainable or can be prepared according to techniques well known to a person skilled in the art. These different elements may be in solid, liquid or gaseous form and may very advantageously be rendered into solution or dissolved in water or any other solvent to be used in the process of the invention. The enzymes used may also be grafted onto a support (in the case of supported enzymes).

According to a preferred embodiment, said compound of formula (II) is O-acetyl-L-homoserine, the enzyme used is O-acetyl-L-homoserine sulfhydrylase and the functionalized mercaptan of formula (I) obtained is L-homocysteine.

The present invention also relates to a composition, preferably an aqueous solution, comprising:
a compound of formula (II) as defined above;
a sulfhydrylase, preferably a sulfhydrylase associated with the compound of formula (II), said sulfhydrylase being as defined above; and
dissolved H₂S, preferably in excess.

Preferably, said composition comprises:
O-acetyl-L-homoserine;
O-acetyl-L-homoserine sulfhydrylase; and
dissolved H₂S, preferably in excess.

Said composition in particular corresponds to the reaction medium as defined above.

The conditions, characteristics and optional additional components are the same as those defined for the reaction medium as defined above.

In particular, the composition according to the invention does not comprise dissolved oxygen.

Preferably, the H₂S is in excess, preferably in molar excess, relative to the compound of formula (II). The H₂S can therefore be in a superstoichiometric amount relative to the amount of the compound of formula (II).

In particular, the molar ratio H₂S/compound of formula (II) is between 1.1 and 20, preferably between 1.1 and 10, preferentially between 2 and 8, for example between 3.5 and 8, and even more preferentially between 3.5 and 5.

The composition may also comprise a cofactor of the sulfhydrylase as defined above.

In particular, the composition according to the invention makes it possible to implement the process according to the invention.

### DESCRIPTION OF THE FIGURES

FIG. 1 shows the yield (%) of the enzymatic synthesis reaction of L-homocysteine after 1 hour of reaction as a function of the partial pressure of H₂S (bar).

The examples which follow make it possible to illustrate the present invention but are not under any circumstances limiting.

### EXAMPLES

The usual definitions of conversion, of selectivity and of yield are as follows:

Conversion=(number of moles of reactant in the initial state−number of moles of reactant remaining after the reaction)/(number of moles of reactant in the initial state)

Selectivity=Number of moles of reactant converted into the desired product/(number of moles of reactant in the initial state−number of moles of reactant remaining after the reaction)

Yield=conversion X selectivity

### Example 1: Enzymatic Preparation of L-Homocysteine from O-Acetyl-L-Homoserine Under Partial Pressure of H₂S

Stage 1: Preparation of O-acetyl-L-homoserine (OAHS)

O-Acetyl-L-homoserine was synthesized from L-homoserine and acetic anhydride according to the protocol described in the works of Sadamu Nagai, “Synthesis of O-acetyl-L-homoserine”, Academic Press (1971), vol. 17, p. 423−424.

Stage 2: Preparation of the Reaction Medium

10 g/l of O-acetyl-L-homoserine originating from stage 1), this product being dissolved in 250 ml of water, are introduced into a thermostatically controlled 500 ml stainless steel reactor. The solution is brought to 37° C. with mechanical stirring.

5 g/l of OAHS Sulfhydrylase and 0.4 g/l of pyridoxal phosphate cofactor are added to the reaction medium so as to reach a total volume of 300 ml. The pH is maintained at a setpoint value of 6.5 using an aqueous ammonia solution (4 M).

The reaction medium is then degassed by nitrogen bubbling for about ten minutes.

Stage 3: Addition of H₂S Under Pressure

The reactor is placed under vacuum in order to eliminate all the gases present in the headspace of the reactor and thus to finely control the pressure of hydrogen sulfide added.

Then a certain pressure of H₂S is applied (PH₂S=Ptotal). The start of the reaction is confirmed by a gradual acidification of the reaction medium (due to the gradual release of the acetic acid coproduct) and the pH of the solution is maintained at around 6.5 via the gradual addition of ammonium hydroxide (4M).

Analysis.

The yield of the reaction is measured after 1 hour of reaction via an approach of quantification of the mercaptan formed by argentometric potentiometric titration (results also confirmed by NMR and HPLC analyses).

Results.

The yield of L-homocysteine after one hour of reaction was determined for several tests with different partial pressures of H₂S in the gas headspace of the reactor used.

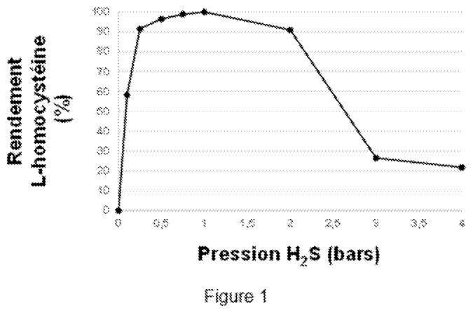

The results show that there are three phases (cf. FIG. 1):
an increase in the yield between 0 and 0.25 bar of partial pressure of H₂S;
a plateau phase, with a yield of between 90% and 100%, between 0.25 and 2 bar of partial pressure of H₂S;
a reduction in this yield for partial pressures of H₂S greater than 2 bar up to 4 bar.

### Example 2: Enzymatic Preparation of L-Homocysteine from O-Acetyl-L-Homoserine Under Partial Pressure of H₂S and in a Non-Degassed Reaction Medium

Stage 1: Preparation of O-Acetyl-L-Homoserine (OAHS)

O-Acetyl-L-homoserine was synthesized from L-homoserine and acetic anhydride according to the protocol described in the works of Sadamu Nagai, “Synthesis of O-acetyl-L-homoserine”, Academic Press (1971), vol. 17, p. 423−424.

Stage 2: Preparation of the Reaction Medium

10 g/l of O-acetyl-L-homoserine originating from stage 1), this product being dissolved in 250 ml of water, are introduced into a thermostatically controlled 500 ml stainless steel reactor. The solution is brought to 37° C. with mechanical stirring.

5 g/l of OAHS Sulfhydrylase and 0.4 g/l of pyridoxal phosphate cofactor are added to the reaction medium so as to reach a total volume of 300 ml. The pH is maintained at a setpoint value of 6.5 using an aqueous ammonia solution (4 M).

Stage 3: Addition of H₂S Under Pressure

The reactor is placed under vacuum in order to eliminate all the gases present in the headspace of the reactor and thus to finely control the pressure of hydrogen sulfide added. Then a pressure of 0.25 bar of H₂S is applied. The start of the reaction is confirmed by a gradual acidification of the reaction medium (due to the gradual release of the acetic acid coproduct) and the pH of the solution is maintained at around 6.5 via the gradual addition of ammonium hydroxide (4M).

Analysis.

The yield of the reaction is measured after 1 hour of reaction via an approach of quantification of the mercaptan formed by argentometric potentiometric titration (results also confirmed by NMR and HPLC analyses).

Results.

Yied of L-homocysteine at Tfinal: 88.4%

1. Process for synthesizing at least one functionalized mercaptan of the following general formula (I): R₂—X—C*H(NR₁R₇)—(CH₂)ₙ—SH (I) in which,
R₁ and R₇, which are identical or different, are a hydrogen atom or an aromatic or nonaromatic, linear, branched or cyclic, saturated or unsaturated, hydrocarbon chain of 1 to 20 carbon atoms which may comprise one or more heteroatoms;
X is chosen from —C(═O)—, —CH₂— or —CN;
R₂ is:
(i) either absent when X represents —CN,
(ii) or a hydrogen atom,
(iii) or —OR₃, R₃ being a hydrogen atom or an aromatic or nonaromatic, linear, branched or cyclic, saturated or unsaturated, hydrocarbon chain of 1 to 20 carbon atoms which may comprise one or more heteroatoms,
(iv) or —NR₄R₅, R₄ and R₅, which are identical or different, being a hydrogen atom or an aromatic or nonaromatic, linear, branched or cyclic, saturated or unsaturated, hydrocarbon chain of 1 to 20 carbon atoms which may comprise one or more heteroatoms;
n is equal to 1 or 2; and * represents an asymmetric carbon;
said process comprising the stages of:
a) provision of at least one compound of the following general formula (II):
R₂—X—C*H(NR₁R₇)—(CH₂)ₙ-G (II)
in which *, R₁, R₂, R₇, X and n are as defined for formula (I) and
G represents either (i) R₆—C(O)—O—, or (ii) (R₇O)(R₈O)—P(O)—O—, or (iii) R₉O—SO₂—O—; with
R₆ being a hydrogen atom or a linear, branched or cyclic, saturated or unsaturated hydrocarbon chain of 1 to 20 carbon atoms which may comprise one or more aromatic groups and may be substituted by one or more groups chosen from —OR₁₀, (═O), —C(O)OR₁₁, —NR₁₂R₁₃;
R₁₀, R₁₁, R₁₂ and R₁₃ being independently chosen from:
H or a linear, branched or cyclic, saturated or unsaturated hydrocarbon chain of 1 to 20 carbon atoms;
R₇ and R₈, which are identical or different, being a proton, an alkali metal, an alkaline earth metal or an ammonium;
R₉ being chosen from a proton, an alkali metal, an alkaline earth metal or an ammonium;
b) provision of H₂S;
c) reaction between said at least one compound of formula (II) and H₂S in the presence of at least one enzyme chosen from sulfhydrylases, preferably a sulfhydrylase associated with said compound of formula (II);
said reaction being performed in a reactor with a partial pressure of H₂S in the gas headspace of said reactor of between 0.01 and 4 bars, for example between 0.01 and 3 bars, preferably between 0.1 and 3 bars, for example between 0.1 and 2.5 bars, at the reaction temperature;
d) obtaining of at least one functionalized mercaptan of formula (I);
e) optional separation of said at least one functionalized mercaptan of formula (I) which is obtained in stage d); and
f) optional additional functionalization and/or optional deprotection of the functionalized mercaptan of formula (I) which is obtained in stage d) or e); and
wherein stages a) and b) are optionally performed simultaneously.

2. Synthesis process according to claim 1, wherein the partial pressure of H₂S in the gas headspace of said reactor is of between 0.25 and 2 bars.

3. Synthesis process according to claim 1, wherein the H₂S is in excess relative to the compound of formula (II).

4. Synthesis process according to claim 1, wherein stage c) is carried out in aqueous solution.

5. Synthesis process according to claim 1, wherein the partial pressure of H₂S corresponds to the total pressure in said gas headspace.

6. Synthesis process according to claim 1, wherein the partial pressure of H₂S is kept constant throughout the entire duration of stage c).

7. Synthesis process according to claim 1, wherein the compound of formula (II) is chosen from the group consisting of:
O-phospho-L-homoserine, O-succinyl-L-homoserine, O-acetyl-L-homoserine, O-acetoacetyl-L-homoserine, O-propio-L-homoserine, O-coumaroyl-L-homoserine, O-malonyl-L-homoserine, O-hydroxymethylglutaryl-L-homoserine, O-pimelyl-L-homoserine and O-sulfato-L-homoserine, preferably O-acetyl-L-homoserine.

8. Synthesis process according to claim 1, wherein the functionalized mercaptan of formula (I) is L-homocysteine.

9. Synthesis process according to claim 1, wherein said compound of formula (II) is O-acetyl-L-homoserine, the enzyme used is O-acetyl-L-homoserine sulfhydrylase and the functionalized mercaptan of formula (I) is L-homocysteine.

10. Process according to claim 1, wherein stage c) is carried out essentially in the absence of oxygen, preferably in the absence of oxygen.

11. Process according to claim 1, wherein the temperature during stage c) is between 10° C. and 60° C., preferably between 20° C. and 40° C., and more particularly between 25° C. and 40° C.

12. Composition comprising:
a compound of formula (II) as defined in claim 1;
a sulfhydrylase; and
dissolved H₂S.
